(19) United States
(12) Patent Application Publication
(10) Pub. No.: US 2010/0056473 A1
(43) Pub. Date: Mar. 4, 2010

(54) METHOD OF EXTRACTING FUCOIDAN

(75) Inventor: Hideaki HAGIWARA, La Jolla, CA (US)

Correspondence Address: WILSON, SONSINI, GOODRICH & ROSATI, 650 PAGE MILL ROAD, PALO ALTO, CA, 94304-1050, US

(73) Assignee: HIHIMSA FOUNDATION, La Jolla, CA (US)

(21) Appl. No.: 12/617,564

(22) Filed: Nov. 12, 2009

Related U.S. Application Data
(62) Division of application No. 11/252,674, filed on Oct. 18, 2005.

(51) Int. Cl. A61K 31/715; C07H 1/00

(52) U.S. Cl. 514/54; 536/123.1

(57) ABSTRACT

A method of extracting fucoidan from a biological material is provided. The method comprises (a) combining a biological material containing fucoidan and a medium to form a mixture; (b) applying ultrasonic waves to the mixture while the mixture is at a temperature of less than about 60 C; (c) extracting fucoidan into the medium from the biological material to produce a fucoidan containing solution and insoluble biological matter; and (d) separating the fucoidan containing solution from the insoluble biological matter to obtain a fucoidan containing extract. Methods of using the fucoidan containing extract and pharmaceutical preparations containing the fucoidan containing extract are also provided.

### BACKGROUND OF THE INVENTION

Fucoidan is a natural product that is obtained as an extract from brown algae such as nemacystus, undaria seaweeds, and sea tangles. Structurally, fucoidan is a sulfated polysaccharide whose primary consitituent sugar is fucose. Fucoidan has become a desirable additive for food, cosmetic, and pharmaceutical preparations based on reports of the various pharmacologic properties of fucoidan. For example, fucoidan has been reported to have antitumor effects (Maruyama, F. et al. Kitasato Arch. Of Exp. Med., 60:105-121 (1987), Ellouali, M. et al., Anticancer Res., 13:2011-2019 (1993)), antiulcer effects (Japanese Patent Publication Nos. 7 [1995]-138166 and 10 [1998]-59860), antivirus effect (Bana, M. et al., Antimicrob. Agents Chemother., 32:1742-1745 (1988), Bana, et al., Antiviral. Res. 9:335-343 (1988), and Clark, G. F. et al., FASEB J., 6:233 (1992)); anti-inflammatory effects (Japanese Patent Publication No. 8 [1996]-92103, Heinzelmann, M. et al., Infect. Immun., 66:5842-5847 (1998), Gan, L. et al., Invest. Ophthalmol. Vis. Sci., 40:575-581 (1999)); anticoagulant effects (Colliec,

S. et al., Phytochemistry, 35:697-700 (1994), Millet, J. et al., Thrombo, Haemost., 81:391-395 (1999)); immunopotentiating effects (Japanese Patent Publication No. 11 [1999]-228602); Type I antiallergic effects (Japanese Patent Publication No. 10 [1998]-72362); and antihyperlipemic effects.

Methods of extracting fucoidan from brown algae including various heat extraction, acidic extraction, and alkaline extraction methods are known. More information on these known methods of extracting fucoidan can be found in Japanese Patent Publication Nos.10 [1998]-195105; 2002-220402; and 2002-262788.

Disadvantageously, however, one or more of these methods of extracting fucoidan can result in low extraction efficiency and/or the extraction of undesirable by-products such as alginic acid, arsenic, and heavy metals along with the desired fucoidan. The removal of these by-products can be difficult, costly, and produce additional chemical by-products that can require costly disposal. For example, the presence of alginic acid in the fucoidan extract makes further refinement of the fucoidan extract, such as ultrafiltration, difficult as alginic acid tends to be highly viscose. In addition, separating co-extracted alginic acid from fucoidan can lower the yield and quality of the refined fucoidan due to the methodology and process involved in removing alginic acid. Known methods of fucoidan extraction at room temperature using acidic or alkaline conditions can require an extended time period to complete the extraction process, which can result in a risk of microorganism contamination, or can require further chemical processing.

In addition, fucoidan extracted from nemacystus using known methods can have a distinct seaweed smell and color reaction, which is an obstacle in adding fucoidan to various types of food and cosmetics.

Therefore, there is a need for a method of extracting fucoidan from algae having a high level of extraction efficiency, a sufficiently low co-extraction level of undesirable by-products, and a resulting fuciodan extraction product that has a reduced seaweed smell and color.

### SUMMARY OF THE INVENTION

According to the present invention, a method of extracting fucoidan from a biological material is provided. Preferably, the biological material is a brown algae, and more preferably the brown algae is nemacystus. The method comprises first combining the biological material containing fucoidan and a medium, in a preferred ratio of about 6 to about 8 (wet wt. to wet wt.). Preferably, the medium is water at a neutral pH of about 7 to form a mixture. Then, ultrasonic waves are applied to the mixture while the mixture is at a temperature of less than about 60° C. The application of ultrasonic waves extracts fucoidan into the medium from the biological material to produce a fucoidan containing solution and insoluble biological matter. The fucoidan containing solution is then separated from the insoluble biological matter to obtain a fucoidan containing extract. In another embodiment of the invention, the method further comprises causing a fucoidan containing solid to separate out from the fucoidan containing solution; and separating the fucoidan containing solid from the fucoidan containing solution to obtain a fucoidan containing solid, preferably in the form of a precipitate. The fucoidan containing extract may be obtained in a purified form by this method, characterized by a neutral sugar content in the coarse fucoidan powder of 33.4%, as converted fucose; and a sulfate content of 20.3%.

In another embodiment, the invention is a method of using a fucoidan containing extract. According to this embodiment, a fucoidan containing extract according to the present invention is provided and used as a nutritional supplement, or an additive for food, cosmetics, or pharmaceutical preparations.

In another embodiment, a composition comprising a pharmaceutical preparation is provided. The composition comprises of an effective amount of a fucoidan containing extract according to the present invention and a pharmaceutically acceptable carrier. The preparation may be in the form of a tablet, capsule, granule, powder, or solution.

In another embodiment, the invention is a method of treating a patient with a disease. According to this embodiment, a disease is diagnosed, or a diagnosis of a disease is confirmed, and a patient is treated with a fucoidan containing extract obtained according to the method according to the present invention. Preferably, the disease is one of a viral, cancerous, inflammatory, or autoimmune disease.

### DETAILED DESCRIPTION OF THE PREFERRED EMBODIMENT

According to one embodiment of the present invention, there is provided a method for extracting fucoidan. The method comprises extracting fucoidan from a plant material such as algae or seaweed by the application of sonic waves. The method achieves the same or a higher level of extraction efficiency than existing hydrothermal extraction methods and the elution of alginic acid, arsenic, and heavy metals is significantly low. Due to the low production of alginic acid during the extraction process, subsequent production processes for extracting fucoidan are easier. Further, it possible to omit subsequent processing steps to remove arsenic and heavy metals. According to the method of the present invention, fucoidan is selectively extracted so that undesirable smell and color, inherent in the plant material, may be removed at the extraction phase. Thus, a fucoidan-containing extract, obtained according to the present invention, may be used as a raw material or further refined and added to food, beverages, cosmetics, and pharmaceutical products.

As used herein, the following terms have the following meanings.

The term “algae” means any of an organism classified into the Protista kingdom and is a chlorophyll-bearing organism that can occur in both salt- and freshwater and reproduce from a unicellular spore and ranges in size from a single cell to giant kelp, and includes most kinds of seaweed.

The term “brown algae” means any of an organism classified into the Protista kingdom and further into the Phaeophyta division.

The term “fucoidan” means a sulfated fucopolysaccharide.

The term “biological material” means any of an organism from the Protista, Fungi, and

Plantae kingdoms in raw (e.g., fresh, frozen, and dried) forms and pre-processed (e.g., pureed, chopped, and ground) forms.

As used in this disclosure, the term “comprise” and variations of the term, such as “comprising” and “comprises,” are not intended to exclude other additives, components, integers or steps.

All amounts disclosed herein are given in weight percent of the total weight of the composition unless otherwise indicated.

In one embodiment, the present invention is a method of extracting fucoidan from a biological material comprising combining the biological material containing fucoidan and a medium to form a mixture.

The biological material may be any organism that contains fucoidan in its natural state and the biological material may be in its raw form such as fresh, frozen, or dried, and/or may also be mechanically pre-processed such as pureed, chopped, or ground. Preferably, the biological material is an algae. More preferably, the biological material is a brown algae. Most preferably, the biological material is the brown algae nemacystus, also referred to as Itomozuku, Mozuka, and Mozuku. The medium is a fluid that can sustain the extracted fucoidan. Preferably, the medium is water, with a pH of between 3.5 to about 10.5. More preferably, the pH of the medium is about 7. However, the medium can be other fluids or mixtures and may contain other ingredients and/or nutrients to aid in the extraction process and sustain the extracted fucoidan.

The ratio of medium to biological material may vary depending on the type, form, and amount of biological material that is used, as well as the medium that is used. Preferably, when the biological material is nemacystus and the medium is water, the ratio of medium to biological material, wet weight to wet weight, is about 6 to about 8 times. However, other ratios of medium to biological material can be used as will be understood by those of skill in the art by reference to this disclosure.

After the biological material and fucoidan are combined, ultrasonic waves are applied to the mixture of biological material and medium. Generally, the mixture is kept at a temperature of less than about 60° C. Preferably, the mixture is kept at a temperature of between about 20° C. to about 60° C. during the extraction. The application of ultrasonic waves extracts fucoidan into the medium from the biological material to produce a fucoidan containing solution and insoluble biological matter. The ultrasonic waves are applied for a time period that is appropriate to extract a sufficient amount of fucoidan from the biological material. Preferably, ultrasonic waves are applied to the mixture of biological material and medium for at least about 0.2 hours. However, the amount of time that the ultrasonic waves are applied to the mixture and the temperature that is used during the extraction may vary, again, depending on the type, form, and amount of biological material that is used in the extraction, as will be understood by those of skill in the art by reference to this disclosure.

After the ultrasonic waves are applied, the fucoidan containing solution is separated from the insoluble biological matter. Generally, this is accomplished first by crude filtration of the mixture through a coarse filter such as nylon mesh, for example. Then, the filtrate, containing the fucoidan extract, may be further processed to remove residual insoluble biological matter. Preferably, the fucoidan extract is centrifuged to remove additional insoluble biological matter. However, other purification processes can be used, such as additional filtration through fine filters, or chromatographic separation as will be understood by those of skill in the art by reference to this disclosure.

In certain embodiments, the fucoidan extract may be further processed to obtain a fucoidan extract in powdered form. According to this embodiment, a fucoidan containing solid is separated from the fucoidan containing solution and then isolated from the residual solution. Preferably, a precipitating agents, such as a potassium acetate solution, followed by ethanol, is added to the fucoidan containing solution, followed by thorough mixing. The resulting fucoidan containing precipitate is then isolated from the solution. The fucoidan containing precipitate can be isolated by known methods such as filtration, and/or centrifugation. Preferably, the fucoidan containing precipitate is isolated by centrifuging the fucoidan containing solution to collect the precipitate. After collecting the precipitate, the fucoidan containing extract (in precipitate form) may then be further purified by washing, such as with ethanol, and then dried (e.g., dessicated) to obtain a fucoidan containing powder.

In another embodiment, the present invention is a use of a fucoidan containing extract as a nutritional supplement. In yet another embodiment, the present invention is the use of a fucoidan containing extract as or as an additive for a food, cosmetic, or pharmaceutical preparation. The pharmaceutical preparation may be in a form such as a tablet, a capsule, granules, a powder, or a solution and comprise other ingredients such as one or more pharmaceutically acceptable carrriers as is known to those of skill in the art.

In other embodiments, the present invention is a method of treating a patient with a disease, where the disease is a viral, cancerous, inflammatory, or autoimmune disease. According to this embodiment, the method comprises diagnosing the patient with the disease, and treating the patient with a fucoidan containing extract, obtained according to the method described herein.

### EXAMPLE I

### Fucoidan Extraction using Ultrasonic Waves

According to one embodiment of the present invention, fucoidan was extracted from brown algae using sonic waves at a temperature below 60° C. as follows. Brown algae (Tongan frozen nemacystus, 15 g) was measured into a 100 ml beaker. Twice the amount of purified water (30 ml) was then added. The beaker was then placed into an ultrasonic cleaner (Bransonic 72, output 375 W, frequency 45 kHz, Branson Ultrasonics Corporation, Danbury, Conn.), which was filled with water. Ultrasonic waves were then applied for 4 hours. The temperature of the water inside the ultrasonic cleaner was increased gradually from 20 C to 48 ° C. during the ultrasonic extraction. After four hours, the nemacystus was filtered through nylon mesh. The filtrate was then centrifuged for 30 minutes at 3500 rpm to remove any insoluble matter. The viscosity of the obtained supernatant (30.5 ml) was observed to be significantly low, indicating no elution of alginic acid. The neutral sugar content of the supernatant, evaluated by the phenol-sulfuric acid method, was 25.5 mg (as converted into fucose).

### EXAMPLE II

### Comparative Example of Fucoidan Extraction at 50 ° C.

Brown algae (frozen nemacystus, 15 g) was measured into a 100 ml beaker. Twice the amount of purified water (30 ml) was then added to the beaker. The beaker was placed into an incubator at 50° C. and was kept warm while the contents were stirred intermittently. After four hours, the nemacystus was filtered using nylon mesh and the filtrate was centrifuged for 30 minutes at 3500 rpm to remove any insoluble matter. The neutral sugar content of the obtained supernatant (30.2 ml) was 11.1 mg (as converted into fucose).

As shown in Examples I and II, fucoidan can be efficiently extracted by the application of ultrasonic waves. The amount of extracted fucoidan (measured as neutral sugar content) using the ultrasonic extraction method of the present invention is approximately 2.3 times higher than the extraction method demonstrated in Example II at 50° C.

### EXAMPLE III

### Fucoidan Extraction from Dried Algae using Ultrasonic Waves

Brown algae (dried nemacystus, 7.2 g) was measured into a 300 ml conical flask. Purified water (290 ml) was then added to the flask. The flask was placed into an ultrasonic cleaner (Bransonic 1200 Model B-1200J-1, output 30 W, frequency 45 kHz, Branson Ultrasonics Corporation, Danbury, Conn.), which was filled with water. Ultrasonic waves were applied to the flask containing the nemacystus for 6 hours. During the ultrasonic extraction, the temperature of the water inside the ultrasonic cleaner was increased gradually from 20° C. up to 48° C. After four hours, the nemacystus was filtered using nylon mesh and the filtrate was processed in a boiling bath for 30 minutes. The filtrate was then centrifuged for 30 minutes at 3500 rpm to remove any insoluble matter. The viscosity of the obtained supernatant (30.5 ml) was significantly low, indicating there was no elution of alginic acid. The neutral sugar content of this supernatant, evaluated by the phenol-sulfuric acid method, was 25.5 mg (as converted into fucose).

### EXAMPLE IV

### Comparitive Example of Fucoidan Extraction from Dried Algae at 95° C.

Brown algae (dried nemacystus, 5 g) was measured into a 300 ml conical flask and 200 ml of purified water was added. The flask was incubated for one hour in a 95° C. bath. After 1 hour, the nemacystus was filtered using nylon mesh and the obtained hot filtrate was left standing until it returned to room temperature. The filtrate at room temperature was viscose due to the eluted alginic acid, and an alcohol precipitation could not be conducted.

### EXAMPLE V

### Fucoidan Extraction Optimization

As described below, to optimize the fucoidan extraction from nemacystus using ultrasonic waves, a comparative study on the amount of purified water added relative to the nemacystus wet weight during the extraction was made.

Using the amounts shown in Table 1, Tongan frozen nemacystus was measured into a 50 ml beaker so that the amount of purified water added during the extraction would be approximately 30 ml (Table 1). Each beaker was placed into an ultrasonic cleaner (Bransonic 72, output 375 W, frequency 45 kHz), which was filled with 50° C. water. Ultrasonic waves were applied for 4 hours while maintaining the water temperature at 50° C. (±4° C.). After 4 hours, the nemacystus was filtered using nylon mesh and the filtrate was centrifuged for 30 minutes at 3500 rpm to remove any insoluble matter. The viscosity of the supernatants obtained from each beaker (30.5 ml) was significantly low, indicating no elution of alginic acid. The neutral sugar content (as converted into fucose) of the supernatant for each sample was evaluated by the phenol-sulfuric acid method. The results are shown in Table 1.

As shown in Table 1, it was found that the yield of neutral sugar extracted from 1 g of frozen nemacystus was more than 0.4% when 6 to 8 times the amount of purified water to nemacystus (wt/wt) was added during the extraction.

TABLE 1

| Ratio of | | | | Yield of neutral |
|---|---|---|---|---|
| Water to wet | Nemacystus | Extracted | Neutral | sugar per 1 g of |
| Nemacystus | wet weight | Supernatant | sugar | nemacystus |
| (wt/wt) | (g) | (ml) | content (mg) | (%) |
| 2 times | 15.37 | 32.5 | 48.43 | 0.315 |
| 4 times | 8.37 | 32.5 | 30.33 | 0.363 |
| 6 times | 5.43 | 30.4 | 23.51 | 0.433 |
| 8 times | 4.05 | 29.8 | 16.39 | 0.405 |

### EXAMPLE VI

### Fucoidan Containing Powdered Extract

According to another embodiment of the present invention, fucoidan was extracted from brown algae and further purified as described below to obtain a fucoidan containing powder.

Brown algae (Tongan frozen nemacystus, 33 g) was measured into a 500 ml beaker. Approximately 6 times a larger amount (wt/wt) of purified water (200 ml) was then added to the beaker. The beaker was then placed into an ultrasonic cleaner (Bransonic 1200 Model B-1200J-1, output 30 W, frequency 45 kHz), which was filled with water. Ultrasonic waves were applied for 24 hours. No particular temperature control was used on the ultrasonic cleaner. However, the water temperature was increased gradually to nearly 50° C., but the temperature was never increased to beyond 60° C. After 24 hours, the nemacystus was filtered using nylon mesh and the filtrate was centrifuged for 30 minutes at 3500 rpm to remove any insoluble matter. The viscosity of the obtained supernatant (210 ml) was significantly low, indicating no elution of alginic acid. Potassium acetate solution (5 ml of 3M, pH 5.2) was added to the supernatant and mixed thoroughly. Ethanol (500 ml) was added to the supernatant to precipitate the fucoidan containing extract. After 4 hours at room temperature, the ethanol solution was centrifuged for 30 minutes to collect the precipitate. The precipitate was cleansed with 80% ethanol and 99.5% ethanol in a vacuum desiccator for drying and hardening. The light brown powder obtained (480 mg, yield per wet weight: 1.45%) was used as a coarse fucoidan powder to measure the neutral sugar content (as converted into fucose) by the phenolsulfate acid method. The sulfate content was measured by the Rhodizonic acid method. The neutral sugar content in the coarse fucoidan powder was 160.3 mg (33.4%) and the sulfate content was measured at 97.5 mg (percentage of sulfate in the entire powder: 20.3%/coarse fucoidan).

As demonstrated by the above results, the sulfate content of the fucoidan extract, obtained by the ultrasonic method of the present invention, is significantly higher than the sulfate content of commercial fucoidan extracted from nemacystus, which is typically 15% -25%/coarse fucoidan. This indicates that the fucoidan is extracted in near-natural condition without being hydrolyzed using the method of the present invention.

Although the present invention has been discussed in considerable detail with reference to certain preferred embodiments, other embodiments are possible. Therefore, the scope of the appended claims should not be limited to the description of preferred embodiments contained herein.

What is claimed is:

1. A method of extracting fucoidan from a biological material comprising:
combining a biological material containing fucoidan and a medium to form a mixture;
applying ultrasonic waves to the mixture while the mixture is at a temperature of less than about 60° C.;
extracting fucoidan into the medium from the biological material to produce a fucoidan containing solution and insoluble biological matter; and
separating the fucoidan containing solution from the insoluble biological matter to obtain a fucoidan containing extract.

2. The method according to claim 1, and further comprising:
causing a fucoidan containing solution to separate out from the fucoidan containing solid; and
separating the fucoidan from the fucoidan containing solid to obtain a fucoidan containing solution.

3. The method according to claim 1, wherein the biological material is brown algae.

4. The method according to claim 3, wherein the biological material is nemacystus.

5. The method according to claim 1, wherein the medium is water.

6. The method according to claim 1, wherein the medium has a pH of about 7.

7. The method according to claim 1, wherein the mass ratio of medium to biological material is from about 6 to about 8.

8. The method according to claim 1, wherein the fucoidan containing extract is characterized by a neutral sugar content in the coarse fucoidan powder of 33.4%, as converted fucose; and a sulfate content of 20.3%.

9. A method of using a fucoidan containing extract comprising:
a) providing a fucoidan containing extract according to claim 1; and
b) using the fucoidan containing extract as a nutritional supplement.

10. A method of using a fucoidan containing extract comprising:
a) providing a fucoidan containing extract according to claim 1; and
b) using the fucoidan containing extract as a food additive.

11. A method of using a fucoidan containing extract comprising:
a) providing a fucoidan containing extract according to claim 1; and
b) using the fucoidan containing extract as a cosmetic additive.

12. A method of using a fucoidan containing extract comprising:
a) providing a fucoidan containing extract according to claim 1; and
b) using the fucoidan containing extract in a pharmaceutical preparation.

13. A composition comprising a pharmaceutical preparation of an effective amount of a fucoidan containing extract according to claim 1 and a pharmaceutically acceptable carrier.

14. The composition of claim 13, wherein the pharmaceutical preparation is in the form of a tablet.

15. The composition of claim 13, wherein the pharmaceutical preparation is in the form of a capsule.

16. The composition of claim 13, wherein the pharmaceutical preparation is in the form of a granule.

17. The composition of claim 13, wherein the pharmaceutical preparation is in the form of a powder.

18. The composition of claim 13, wherein the pharmaceutical preparation is in the form of a solution.

19. A method of treating a patient with a disease comprising the steps of:
a) diagnosing the disease or confirming a diagnosis of the disease; and
b) treating the patient with a fucoidan containing extract according to claim 1.

20. The method of claim 19, wherein the disease is one of a viral, cancerous, inflammatory, or autoimmune disease.

21. A fucoidan containing extract characterized by a neutral sugar content in the coarse fucoidan powder of 33.4%, as converted fucose; and a sulfate content of 20.3%.
